(19) United States
(12) Patent Application Publication
(10) Pub. No.: US 2014/0194742 A1
(43) Pub. Date: Jul. 10, 2014

(54) ULTRASOUND IMAGING SYSTEM AND METHOD

(71) Applicant: General Electric Company, Schenectady, NY (US)

(72) Inventors: Subin Sundaran Baby Sarojam, Bangalore (IN); Mohandas Vasudevan, Bangalore (IN)

(73) Assignee: General Electric Company, Schenectady, NY (US)

(21) Appl. No.: 14/136,166

(22) Filed: Dec. 20, 2013

(30) Foreign Application Priority Data
Dec. 28, 2012 (IN) 5488/CHE/2012

(51) Int. Cl. A61B 8/00; A61B 8/08

(52) U.S. Cl. CPC A61B 8/54; A61B 8/461; A61B 8/52; A61B 8/4466; A61B 8/4494; A61B 8/4427
USPC 600/459; 600/437

(57) ABSTRACT

An ultrasound imaging system and method includes performing a gesture with a scan system and detecting the gesture based on data from a motion sensing system in the scan system. The motion sensing system includes at least one sensor selected from the group of an accelerometer, a gyro sensor and a magnetic sensor. The ultrasound imaging system and method also includes performing a control operation based on the detected gesture.

### FIELD OF THE INVENTION

This disclosure relates generally to an ultrasound imaging system and a method for performing a control operation based on a gesture performed with a scan system.

### BACKGROUND OF THE INVENTION

Conventional hand-held ultrasound imaging systems typically include a probe and a scan system. The probe contains one or more transducer elements that are used to transmit and receive ultrasound energy. The controls used to control the hand-held ultrasound imaging system are typically located on the scan system. For example, the user may control functions such as selecting a mode, adjusting a parameter, or selecting a measurement point based on control inputs applied to the scan system. Some conventional hand-held ultrasound imaging systems use touch screens as part or all of the user interface. When using a hand-held ultrasound imaging system, both of the user's hands are typically occupied. For example, a user would typically hold the probe in one hand while holding the scan system in their other hand. Since both hands are occupied while scanning with a typical hand-held ultrasound imaging system, it can be difficult for the user to perform various control operations. Further, for ultrasound scanning a small angle in the probe side makes a significant difference in the details of the target/organ. Most often making these small changes in the angle or movement at the probe side is a challenge. This could involve lots of human errors and is a time consuming activity. This will be challenging for a person who is not well versed in performing scans. Thus the imaging process can be simplified, if any assistance in maneuvering this small angle or movement of the probe is provided.

For these and other reasons an improved ultrasound imaging system and an improved method for controlling an ultrasound imaging system are desired.

### BRIEF DESCRIPTION OF THE INVENTION

The above-mentioned shortcomings, disadvantages and problems are addressed herein which will be understood by reading and understanding the following specification.

In an embodiment, a method of controlling an ultrasound imaging system is disclosed. The method comprises: operating the imaging system is a selected mode of operation; performing a gesture with a scan system; detecting the gesture based on data from a motion sensing system in the scan system, wherein the motion sensing system includes at least one sensor selected from the group comprising of an accelerometer, a gyro sensor, and a magnetic sensor; and performing at least one control operation of the imaging system based on the detected gesture in each mode of operation of the imaging system.

In an embodiment, a method of controlling an ultrasound imaging system is disclosed. The method comprises: inputting a command to select a mode of operation; displaying an image on a scan system; and performing a gesture with the scan system. The gestures of the scan system being detected based on data from a motion sensing system associated with the scan system, wherein the motion sensing system includes at least one sensor selected from a group comprising of an accelerometer, a gyro sensor, and a magnetic sensor. Method further comprises: maneuvering a probe based on the detected gesture; and acquiring image data by maneuvering the probe.

In an embodiment, an ultrasound imaging system is disclosed. The imaging system comprises a probe. The probe comprises: a movable head; at least one transducer element disposed in the head; and a motion control system configured to control at least the head or the beam generator. The imaging system further comprises a scan system in communication with the probe. The scan system comprises: a housing; a display; a motion sensing system attachable to the display or to the housing; and a processor, wherein the processor is configured to receive data from the motion sensing system and to interpret the data as a gesture, and translate the gestures to probe control instructions in a first mode of operation of the imaging system.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a schematic representation of an ultrasound imaging system in accordance with an embodiment;

FIG. 3A and FIG. 3B are schematic representations of the front and back views of a scan system in accordance with an embodiment;

FIG. 4 is a schematic representation of a scan system in accordance with an embodiment;

FIG. 5 is a schematic representation of a scan system in accordance with an embodiment;

FIG. 6 is a schematic representation of a hand-held ultrasound imaging system in accordance with an embodiment;

FIG. 7 is schematic representation of a scan system overlaid on a Cartesian coordinate system in accordance with an embodiment;

FIG. 8 shows a method of controlling an ultrasound imaging system in accordance with an embodiment ;and

FIG. 9 shows a method of controlling an ultrasound imaging system in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

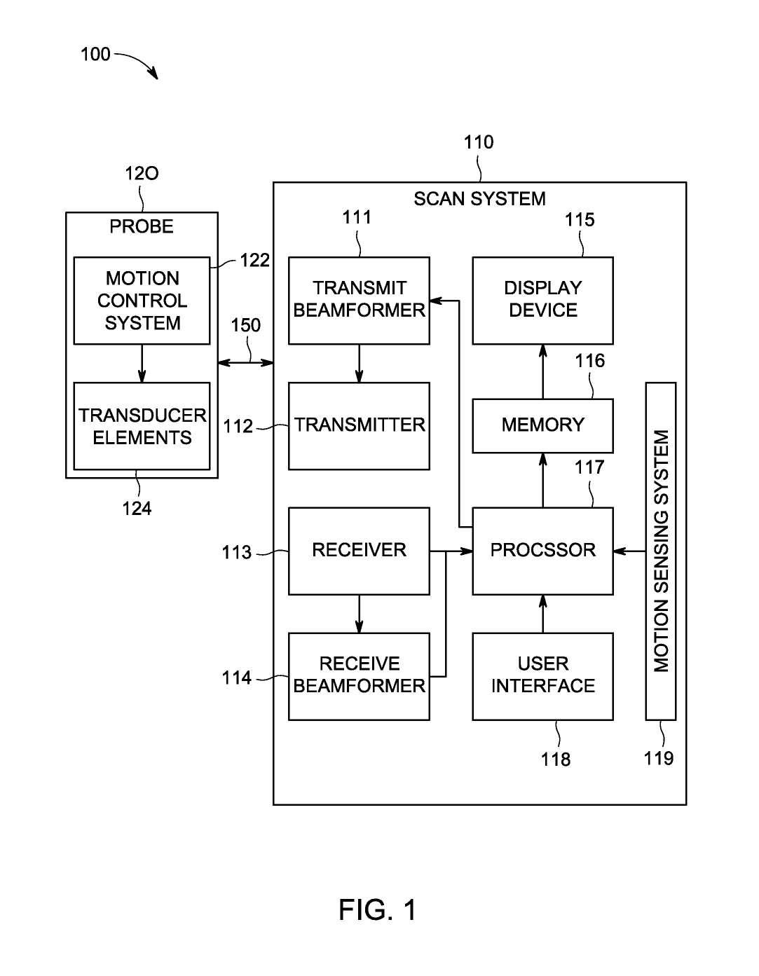

FIG. 1 is a schematic diagram of an ultrasound imaging system 100 in accordance with an embodiment. The ultrasound imaging system includes a scan system 110. According to an exemplary embodiment, the scan system 110 may be a hand-held device. For example, the scan system 110 may be similar in size to a smartphone, a personal digital assistant or a tablet. According to other embodiments, the scan system 110 may be configured as a laptop or cart-based system. The ultrasound imaging system 100 includes a transmit beamformer 111 and a transmitter 112 that drive transducer elements 124 within a probe 120, to emit pulsed ultrasonic signals into an area of a body that is being imaged (not shown). The scan system 110 also includes a motion sensing system 119 in accordance with an embodiment. The motion sensing system 119 may include one or more of the following sensors: a gyro sensor, an accelerometer, and a magnetic sensor. The motion sensing system 119 is adapted to determine the position and orientation of the scan system 119, preferably in real-time, as a clinician is performing the imaging operation using the probe 120. For purposes of this disclosure, the term “real-time” is defined to include an operation or procedure that is performed without any intentional delay. In an alternate embodiment, the motion sensing system 119 is adapted to determine the position and orientation of the scan system 119, preferably in real-time, as a clinician is processing the images or the image data is being acquired by the imaging system 100. The scan system 110 is in communication with the probe 120. The scan system 110 may be physically connected to the probe 120, or the scan system 110 may be in communication with the probe 120 via a wireless communication technique. The wired or wireless communication channel is shown as 150 in FIG. 1. Still referring to FIG. 1, the pulsed ultrasonic signals are back-scattered from structures in the body, like blood cells or muscular tissue, to produce echoes that return to the elements 124. The echoes are converted into electrical signals, or ultrasound data, by the elements 124 and the electrical signals are received by a receiver 113. The electrical signals representing the received echoes are passed through a receive beamformer 114 that outputs ultrasound data. According to some embodiments, the probe 120 may contain electronic circuitry to do all or part of the transmit and/or the receive beamforming. For example, all or part of the transmit beamformer 111, the transmitter 112, the receiver 113 and the receive beamformer 114 may be situated within the probe 120. The terms “scan” or “scanning” may also be used in this disclosure to refer to acquiring data through the process of transmitting and receiving ultrasonic signals. The terms “data” or “ultrasound data” may be used in this disclosure to refer to either one or more datasets acquired with an ultrasound imaging system. A user interface 118 may be used to control operation of the ultrasound imaging system 100, including, to control the probe 120, to control the input of patient data, to change a scanning or display parameter, and the like. The user interface 118 may include one or more of the following: a rotary knob, a keyboard, a mouse, a trackball, a track pad, and a touch screen. In an embodiment, the user interface 118 is a graphical user interface.

The ultrasound imaging system 100 also includes a processor 117 to control the transmit beamformer 111, the transmitter 112, the receiver 113 and the receive beamformer 114. The processor 117 is in communication with the probe 120, through the communication channel 150. The processor 117 may control the probe 120 to acquire ultrasound data. The processor 117 controls which of the elements 124 are active and the shape of a beam emitted from the probe 120. The processor 117 is also in communication with a display device 115, and the processor 117 may process the data into images for display on the display device 115. According to other embodiments, part or all of the display device 115 may be used as the user interface. For example, some or all of the display device 115 may be enabled as a touch screen or a multi-touch screen. For purposes of this disclosure, the phrase “in communication” may be defined to include both wired and wireless connections.

In an embodiment, the motion sensing system 119 provided along with the scan system 110 is used to detect the position and orientation of the scan system 110. The motion sensing system 119 may be disposed within the scan system 110 or could be detachably associated with the scan system 110.

In an embodiment, the motion sensing system 119 is configured to capture the gestures of the scan system 110. The gestures of the scan system include any linear or rotational movement on the scan system 110. The movements of the scan system/gestures are identified by the motion sensing system 119 and communicated to the processor 117 for further processing. The gestures of the scan system 110 can be used to control the movement of the probe 120 in an image acquisition mode and can be used to control the processing of the image in an image processing mode of operation of the imaging system.

The processor 117 may include a central processor (CPU) according to an embodiment. According to other embodiments, the processor 117 may include other electronic components capable of carrying out processing functions, such as a digital signal processor, a field-programmable gate array (FPGA) or a graphic board. According to other embodiments, the processor 117 may include multiple electronic components capable of carrying out processing functions. For example, the processor 117 may include two or more electronic components selected from a list of electronic components including: a central processor, a digital signal processor, a field-programmable gate array, and a graphic board. According to another embodiment, the processor 117 may also include a complex demodulator (not shown) that demodulates the RF data and generates raw data. In another embodiment the demodulation can be carried out earlier in the processing chain. The processor 117 may be adapted to perform one or more processing operations according to a plurality of selectable ultrasound modalities on the data. The data may be processed in real-time during a scanning session as the echo signals are received. Some embodiments of the invention may include multiple processors (not shown) to handle the processing tasks. For example, a first processor may be utilized to demodulate and decimate the RF signal while a second processor may be used to further process the data prior to displaying an image. It should be appreciated that other embodiments may use a different arrangement of processors.

In an embodiment, the processor 117 is configured to receive data from motion sensing system 119 and process the same. The gestures of the scan system 110 is identified by the motion sensing system 119, the corresponding data preferably in terms of position and orientation of the scan system 110 is communicated to the processor 117. Alternately, the motion sensing system 119 detects the position and orientation of the scan system and based on the same processor identifies the gestures of the scan system. In an image acquisition mode of operation, the processor 117 maps this data to corresponding probe control instructions. In an exemplary embodiment, movement of the scan system 110 by 10 cm towards the user could be translated to a 1 millimeter movement of the probe 120 towards right side. There could be set of control instructions defined based on the movement of the scan system 110. Thus in an image acquisition mode of the imaging system, the movement of the scan system 110 is used to control the movement of the probe 120. Larger movements by the scan system could be converted to corresponding smaller movements at the probe level.

In an image processing mode of the imaging system 100, the images acquired are processed. In this mode, the motion sensing system 119 can be used to detect the gestures of the scan system 110. The gestures of the scan system 110 can be translated to various image processing or user input instructions. For example various gestures could be used to select the desired area in the image, annotate the image, generate volumetric images, change the processing parameters etc.

In an embodiment, the gestures can be used control operations of the scan system. For examples, the gestures of the scan system 110 such as flick, up or down movement, holding the scan system 110 without any movement for some time could be defined to perform some instructions such as print, save, freeze, rotate, zoom etc. The examples need not be limited to these. Any of the gestures of the scan system 110 could be identified and translated to image processing instructions in the image processing mode of operation of the imaging system.

The ultrasound imaging system 100 may continuously acquire data at a frame rate of, for example, 10 Hz to 50 Hz. Images generated from the data may be refreshed at a similar rate. Other embodiments may acquire and display data at different rates. A memory 116 is included for storing processed frames of acquired data. In an embodiment, the predefined user or image processing functions, corresponding to various gestures of the scan system 110, could be stored in the memory 116. A look up table, or any other data, could be stored in memory, which will assist the processor in mapping scan system movements to corresponding probe control instructions including probe movements or image processing instructions. In an embodiment, image processing instructions could include scan system control instructions as well. The memory 116 may comprise any known data storage medium.

In an embodiment, the probe 120 is provided with a motion control system 122 configured to control the probe 120 based on the instructions received from the processor 117. The motion control system 122 may be disposed within the probe or could be detachably associated with the probe 120. In an embodiment, the motion control system 122 is configured to control the movement of the head of the probe based on the control instructions. Alternately, the control instructions can be used to control the beam movement or shape by controlling transducer elements 124. The motion control system 122 includes motors or any other moving mechanism. In an embodiment, the probe 120 may be provided with a display (not shown) in addition to, or by replacing, the motion control system 122. The user could be communicated with the probe control instructions through the display and the instructions could be performed by the user instead of the motion control system 119. In an embodiment, “Beam Steering” technology for steering the ultrasound beam at an angle can be used to control the direction of the beam based on the probe control instructions generated using the scan system gestures. In this event, the motion control system 119 may not be required to control the probe or the beam movement.

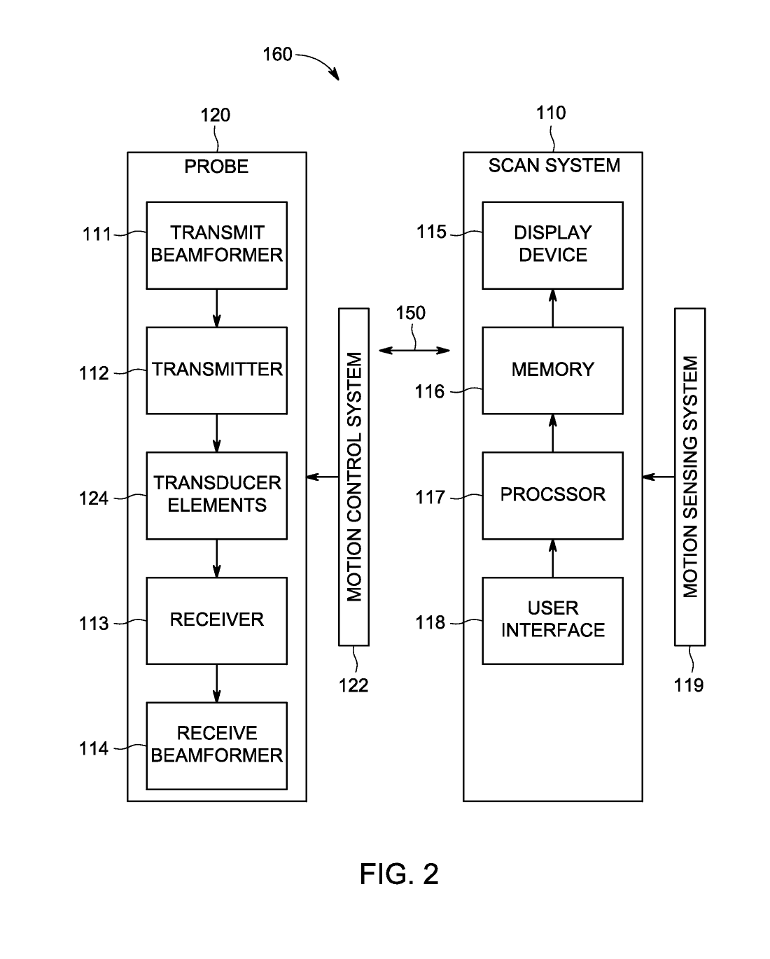

FIG. 2 is a schematic representation of an ultrasound imaging system 100 in accordance with another embodiment. The ultrasound imaging system 100 includes the same components as the ultrasound imaging system described with reference to FIG. 1, but the components are arranged differently. Common reference numbers are used to identify identical components within this disclosure. A probe 120 includes the transmit beamformer 111, the transmitter 112, the receiver 113 and the beamformer 114 in addition to the transducer elements 124. The probe 120 is in communication with a scan system 110. The probe 120 and the scan system 110 may be physically connected, such as through a cable, or they may be in communication through a wireless technique. The communication channel is represented as 150. The elements in the ultrasound imaging system shown in FIG. 2 may interact with each other in the same manner as that previously described for the ultrasound imaging system 100 (shown in FIG. 1). The processor 117 may control the transmit beamformer 111 and the transmitter 112, which in turn, control the firing of the transducer elements 124. The motion sensing system 119 may detect the gestures of the scan system 110, and the processor 117 may generate control instructions to control the probe operation. A motion control system 122 associated with the probe may facilitate implementing these control instructions. Additionally, the receiver 113 and the receive beamformer 114 may send data from the transducer elements 124 back to the processor 117 for processing. The display device 115, memory 116 and user interface 118 shown in FIG. 2, perform substantially the same function as those in FIG. 1. In the embodiment shown in FIG. 2, the motion sensing system 119 and the motion control system 122 are detachably attached to the housing of the scan system 110 and the probe 120 respectively.

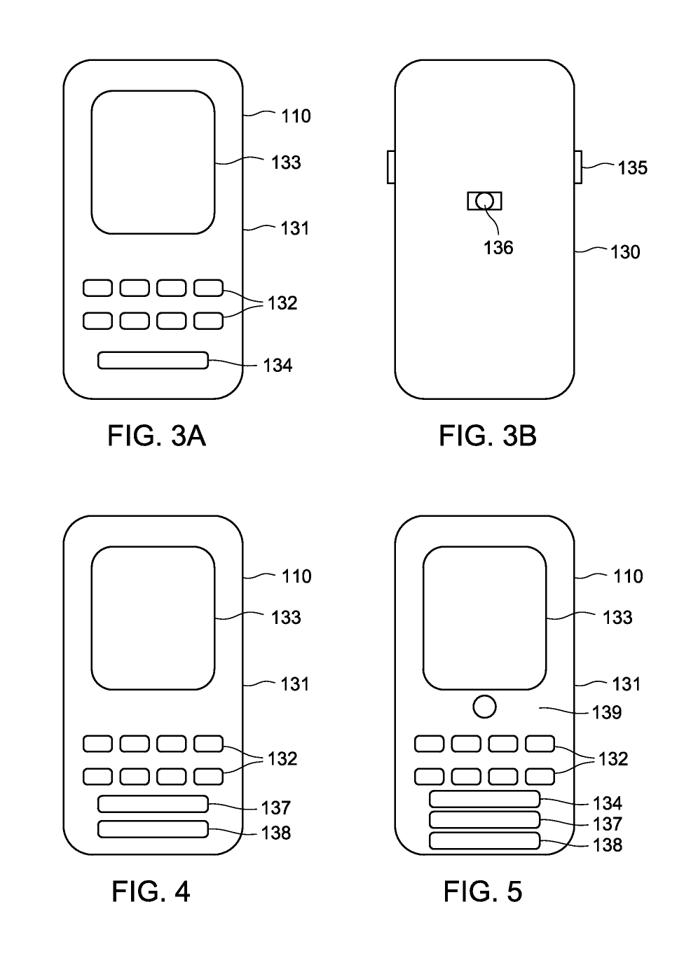

FIGS. 3, 4, and 5 are schematic representations showing additional details of the probe 106 (shown in FIG. 1) in accordance with different embodiments. Common reference numbers will be used to identify identical elements in FIGS. 3, 4, and 5. Structures that were described previously may not be described in detail with respect to FIGS. 3, 4, and 5.

Referring to FIG. 3A, the scanning system 110 includes a housing 131. The motion sensing system includes a magnetic sensor 134. The magnetic sensor 134 could be disposed on the housing 131. The magnetic sensor 134 will be described in detail hereinafter. According to other embodiments, the motion sensing system may include an accelerometer (not shown) or a gyro sensor (not shown) in place of the magnetic sensor 134. The pair of keys 132 are provided, which could be the user interface. The system is provided with a display device, which could be a graphical user interface 133. The scan system 110 is provided with a magnetic sensor 134, which will detect the position and orientation of the scan system. This data is translated to probe control instructions and communicated to the probe (shown in FIG. 1). FIG. 3B shows the back view of the scan system. In an embodiment, control buttons 135 are provided to directly control the probe movement. A user can give instructions to control the probe directly. These instructions could be processed and communicated to the probe directly. Alternately, control buttons 135 could be used to control the image processing. In an embodiment, a track ball/track pad 136 is provided to control the probe or the image processing operation. The location of the control buttons 135 or the track ball 136 is positioned such that the clinician can easily operate the same, even while holding the scan system in one hand and the probe in other hand.

In an embodiment, the actions performed by track ball/pad 136 or control buttons 135 to can be translated into probe control instructions. In an embodiment the control buttons 135 could be used to control the linear motion of the probe. For example, the control buttons 135 could be divided into two parts and each part could be configured to certain predefined movement of the probe. Similarly the track ball 136 movements can also be converted to angular or linear movement of the probe. The pair of control buttons 135 may optionally be used to control image processing or interact with a graphical user interface (GUI) on the display device 133.

The track ball 136 or the control buttons 135 may be positioned elsewhere on the scan system 110 in other embodiments. Each one of the pair of buttons 135 may be assigned a different function so that the user may implement either a “left click” or “right click” to access different functionality through the GUI. Other embodiments may not include the pair of buttons 135. Instead, the user may provide instruction and interact with the GUI through any other interfacing devices which are connectable to the scan system.

The magnetic sensor 134 may include three coils disposed so each coil is mutually orthogonal to the other two coils. For example, a first coil may be disposed in an x-y plane, a second coil maybe disposed in a x-z plane, and a third coil may be disposed in a y-z plane. The coils of the magnetic sensor 134 may be tuned to be sensitive to the strength and direction of a magnetic field that is external to the magnetic sensor 134. For example, the magnet field may be generated by a combination of the earth's magnetic field and/or another magnetic field generator. By detecting magnetic field strength and direction data from each of the three coils in the magnetic sensor 134, the processor 117 (shown in FIG. 1) may be able to determine the absolute position and orientation of the scan system 110. According to an exemplary embodiment, the magnetic field generator may include either a permanent magnet or an electromagnet placed externally to scan system 110. For example, the magnetic field generator may be a component of the scan system 110 (shown in FIG. 1).

FIG. 4 is a schematic representation of the scan system 110 in accordance with another embodiment. Referring to FIG. 4, the scanning system includes a housing 131. The motion sensing system 119 includes an accelerometer 137. The accelerometer 137 may be a 3-axis accelerometer, adapted to detect acceleration in any of three orthogonal directions. For example, a first axis of the accelerometer may be disposed in an x-direction, a second axis may be disposed in a y-direction, and a third axis may be disposed in a z-direction. By combining signals from each of the three axes, the accelerometer 137 may be able to detect accelerations in any three-dimensional direction. By integrating accelerations occurring over a period of time, the processor 117 (shown in FIG. 1) may generate an accurate real-time velocity and position of the accelerometer 137, and hence scan system 110, based on data from the accelerometer 137. According to other embodiments, the accelerometer 137 may include any type of device configured to detect acceleration by the measurement of force in specific directions. The motion sensing system 119 could include a gyro sensor 138. The gyro sensor 138 is configured to detect changes angular velocities and changes in angular momentum, and it may be used to determine angular position information of scan system 110. The gyro sensor 138 may detect rotations about any arbitrary axis. The gyro sensor 138 may by a vibration gyro, a fiber optic gyro or any other type of sensor adapted to detect rotation or change in angular momentum.

FIG. 5 is a schematic representation of the scan system in accordance with another embodiment. The scan system includes motion sensing system 119. The motion sensing system includes a magnetic sensor 134, an accelerometer 137 or a gyro sensor 138. The motion sensing system 119 may additionally include a camera 139, which could detect the position and orientation of the probe or the scan system. The camera 139 could also be used to detect the gestures performed by the user using the scan system and communicate the same to the processor 117. In an example, the ZoomIn/ZoomOut functionality of images can be achieved with the gestures detected by camera. When the scan system is moved towards the face the image can be zoomed out and when it is moved away the images can be zoomed in. Referring now to FIGS. 1, 4, and 5, the combination of data from the gyro sensor 137 and the accelerometer 138 may be used by the processor 117 for calculating the position, orientation, and velocity of the probe 120 without the need for an external reference. The motion sensing system 119 may be used to detect many different types of motion. For example, the motion sensing system 119 may be used to detect translations, such as moving the scan system 110 up and down (also referred to as heaving), moving the scan system 110 left and right (also referred to as swaying), and moving the scan system 110 forward and backward (also referred to as surging). Additionally, the motion sensing system 119 may be used to detect rotations, such as tilting the scan system 110 forward and backward (also referred to as pitching), turning the scan system 110 left and right (also referred to as yawing), and tilting the scan system 110 from side to side (also referred to as rolling).

By tracking the linear acceleration with an accelerometer 137, the processor 117 may calculate the linear acceleration of the scan system 110 in an inertial reference frame. Performing an integration on the inertial accelerations and using the original velocity as the initial condition, enables the processor 117 to calculate the inertial velocities of the scan system 110. Performing an additional integration and using the original position as the initial condition allows the processor 117 to calculate the inertial position of the scan system 110. The processor 117 may also measure the angular velocities and angular acceleration of the scan system 110 using the data from the gyro sensor 139. The processor 117 may, for example, use the original orientation of the scan system 110 as an initial condition and integrate the changes in angular velocity of the scan system 110, as measured by the gyro sensor 146, to calculate the probe's 106 angular velocity and angular position at any specific time. With regularly sampled data from the accelerometer 138 and the gyro sensor 139, the processor 117 may compute the position and orientation of the scan system 110 at any time. From the identified position and orientation of the scan system 110, corresponding position and orientation is derived and communicated to the probe or motion control system 119 associated with the probe 120.

The exemplary embodiment of the scan system 110 shown in FIG. 5 is particularly accurate for tracking the position and orientation of scan system 110 due to the synergy between the attributes of the different sensor types. For example, the accelerometer 137 is capable of detecting translations of the scan system 110 with a high degree of precision. However, the accelerometer 137 is not well-suited for detecting angular rotations of the scan system 110. The gyro sensor 138, meanwhile, is extremely well-suited for detecting the angle of scan system 110 and/or detecting changes in angular momentum resulting from rotating scan system 110 in any arbitrary direction. Pairing the accelerometer 137 with the gyro sensor 138 is appropriate because together, they are adapted to provide very precise information on both the translation of scan system 110 and the orientation of scan system 110. However, one drawback of both the accelerometer 137 and the gyro sensor 138 is that both sensor types are prone to “drift” over time. Drift refers to intrinsic error in a measurement over time. The magnetic sensor 134 allows for the detection of an absolute location in space with better accuracy than just the combination of the accelerometer 137 and the gyro sensor 138. Even though the position information from the magnetic sensor 134 may be relatively low in precision, the data from the magnetic sensor 134 may be used to correct for systematic drifts present in the data measured by one or both of the accelerometer 137 and the gyro sensor 138. Each of the sensor types in scan system 110 shown in FIG. 5 has a unique set of strengths and weaknesses. However, by packaging all three sensor types in scan system 110, the position and orientation of the scan system 110 may be determined with enhanced accuracy and precision.

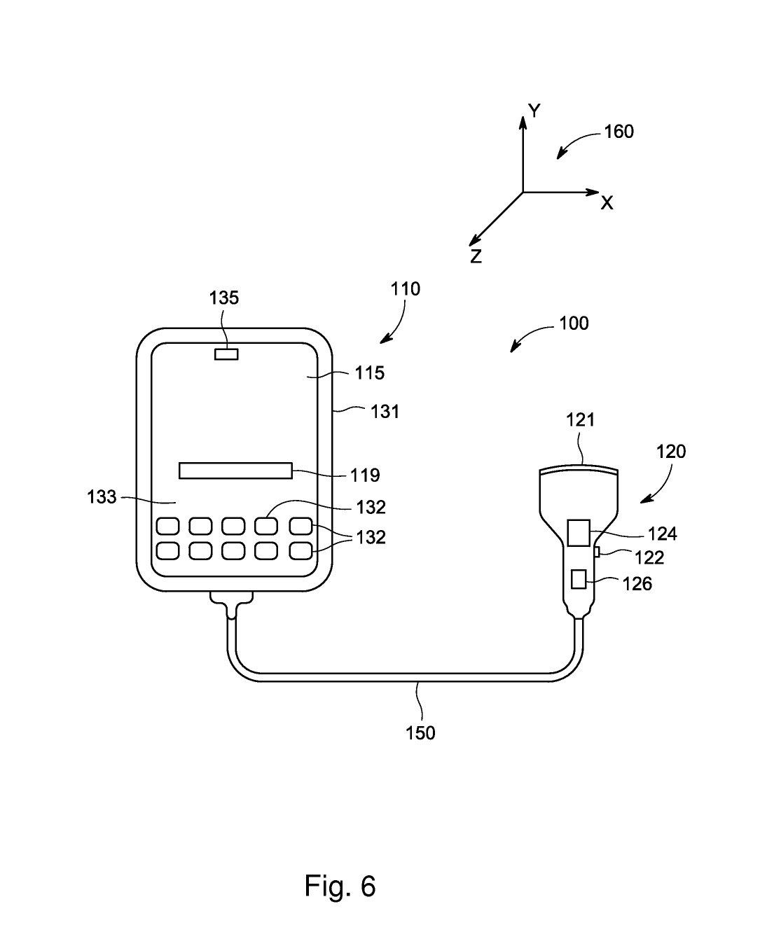

FIG. 6 is a schematic representation of a hand-held or hand-carried ultrasound imaging system 100 in accordance with an embodiment. Ultrasound imaging system 100 includes the scan system 110 and the probe 120 connected by a cable 150 in accordance with an embodiment. According to other embodiments, the probe 120 may be in wireless communication with the scan system 110. The scan system 110 includes the motion sensing system 119. The motion sensing system 119 may, for example, be in accordance with any of the embodiments described with respect to FIG. 3, 4 or 5. The scan system 110 includes the display device 115, which may include an LCD screen, an LED screen, or any other type of display. In an embodiment, the display device 115 may include a graphical user interface 133. Coordinate system 160 includes three vectors indicating an x-direction, a y-direction, and a z-direction. The coordinates system 160 may be defined with respect to the room. For example, the y-direction may be defined as vertical and the x-direction may be defined as being with respect to a first compass direction while the z-axis may be defined with respect to a second compass direction. The orientation of the coordinate system 160 may be defined with respect to the scan system 110 according to other embodiments. For example, according to an exemplary embodiment, the orientation of the coordinate system 160 may be adjusted in real-time so that it is always in the same relationship with respect to the graphical user interface 133. According to an embodiment, the x-y plane, defined by the x-direction and the y-direction of the coordinate system 160 may always be oriented so that it is parallel to a viewing surface of the graphical user interface 133. According to other embodiments, the clinician may manually set the orientation of the coordinate system 160.

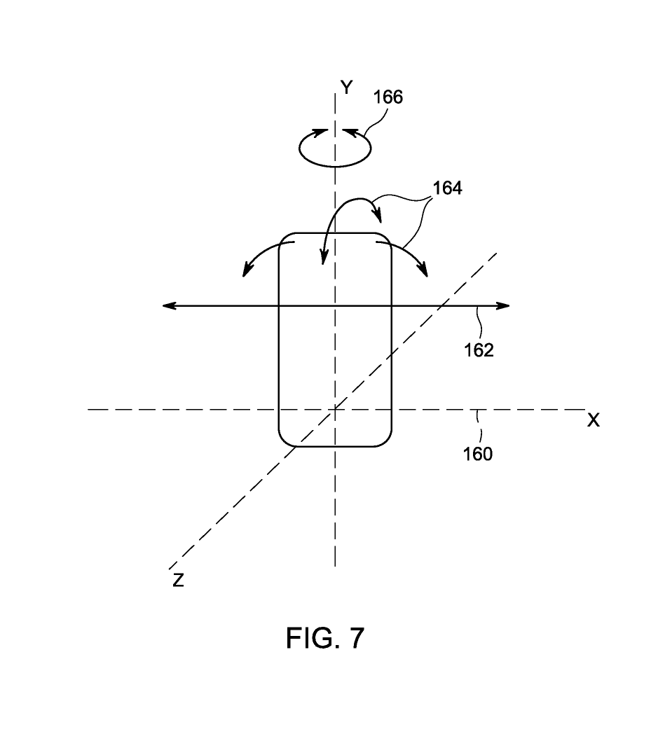

FIG. 7 is a schematic representation of the scan system 110 overlaid on a Cartesian coordinate system 160. The motion sensing system 119 (shown in FIG. 6) may detect the position and orientation of the scan system 110 in real-time, in accordance with an embodiment. Based on data from the motion sensing system 119, the processor 117 (shown in FIG. 1) may determine exactly how the probe 120 can be manipulated. Based on the data from the motion sensing system 119, the processor 117 may also detect any number of gestures, or specific patterns of movement, performed by the user with the scan system 110. The scan system 110 may be translated as indicated by path 162, the scan system 110 may be tilted as indicated by paths 164, and the scan system 110 may be rotated as indicated by path 166. It should be appreciated by those skilled in the art that the paths 162, 164, and 166 represent a limited subset of all the gestures which may be performed with the scan system 110 and detected with the motion sensing system 119. By combining data from the motion sensing system 119 to identifying translations, tilt, and rotations, the processor 117 may detect any gesture performed with the scan system 110 in three-dimensional space.

Referring to FIG. 6, gestures performed with the scan system 110 may be used for a variety of purposes including performing the control operations of the probe. It may be necessary to first input a command to select or activate a specific mode. For example, when activated, the mode may use gestures performed with scan system 110 to control probe movements or control the image processing. According to an embodiment, the clinician may input the command to activate a particular mode by performing a very specific gesture that is unlikely to be accidentally performed during the process of handling scan system 110 or scanning a patient. A non-limiting list of gestures that may be used to select the mode includes moving the scan system 110 in a back-and-forth motion or performing a flicking motion with the scan system 110. In an embodiment, keeping the probe on a target area for imaging could be used as an input to select the mode of operation. The scan system can be operated in an image acquisition mode and an image processing mode. In an embodiment, the gesture preformed with the scan system during the image acquisition mode is used to manipulate the probe movement and the gestures performed with the scan system during image processing mode are used to control the processing of the image data acquired by the scans system 110 in the image acquisition mode. According to other embodiments, the clinician may select a control or switch on scan system 110 in order to toggle between different modes.

According to other embodiments, in the image processing mode, the processor 117 may be configured to perform multiple control operations in response to a single gesture performed with the scan system 110. For example, the processor 117 may perform a series of control operations that are all part of a script, or sequence of commands. The script may include multiple control operations that are commonly performed in a sequence, or the script may include multiple control operations that need to be performed in a sequence as part of a specific procedure. For example, the processor 117 may be configured to detect a gesture and then perform both a control operation and a second control operation in response to the gesture. Additionally, according to other embodiments, a single gesture may be associated with two or more different control operations depending upon the mode of operation of the ultrasound imaging system 100. A gesture may be associated with a first control operation in a first mode of operation and the same gesture may be associated with a second control operation in a second mode of operation. For example, a gesture may be associated with a control operation such as “move” in a first mode of operation, while the same gesture may be associated with a second control operation such as “archive” or “freeze” in a second mode of operation. It should be appreciated that a single gesture could be associated with many different control operations depending on the mode of operation.

In an embodiment, in the image acquisition mode, the processor 117 translates the position or orientation of the scan system 110 to desired/corresponding position and orientation of the probe. The desired position and orientation or the control instructions to achieve desired position and orientation are communicated to the probe 120. The probe or the motion control system 122 associated with the probe 120 receives the desired position and orientation and moves the probe head or adjusts the beam orientation to achieve the desired position or orientation. The desired position can be achieved by the motion control system by adjusting the probe position automatically to the desired position. Alternately, the desired position and orientation can be displayed on the probe 120 and the user can maneuver the probe 120 manually.

According to another embodiment, the gestures of the scan system may be used to process the images or image data acquired during the image acquisition mode. In the image processing mode, the gestures performed by the scan system or the position and orientation of the scan system can be used to control various processing steps. Certain gestures by the scan system can be defined as certain actions or use inputs to perform different steps during image processing. In an example, during image processing it may be desirable to control zooming of the images with gestures from the scan system 110. For example, the clinician may zoom in on the image by moving the scan system 110 further away from the clinician in the z-direction and the clinician may zoom out by moving scan system 110 closer to the clinician in the z-direction. According to other embodiments, the gestures controlling the zoom-in and zoom-out functions may be reversed. By performing gestures with scan system 110 in 3D space, the user may therefore simultaneously control both the zoom of the image displayed on the user interface 133.

Still referring to FIG. 6, an example of a GUI 133 is shown on the display device 115. The GUI 133 could include a menu 135 for the user to select various options. The user could select control instructions for probe or could select the image processing instructions. The GUI 133 also includes a plurality of soft keys 132 or icons, each controlling an image parameter, a scan function, or another selectable feature.

In an embodiment, during image acquisition mode, the scan system gestures could be used to control the movement of probe head 121 or control the operation of transducer elements 124.

In an embodiment, the probe may be provided with a display 126. The probe control instructions including position and orientation information could be provided on this display 126. Based on the displayed instructions, the user could control the probe with or without the assistance of the motion control system 122.

According to another exemplary embodiment, in an image processing mode, the clinician may select an icon or select an operation by performing a flicking motion with scan system 110. The flicking motion may, for instance, include a relatively rapid rotation in a first direction and then a rotation back in the opposite direction. The user may perform either the back-and-forth motion or the flicking motion relatively quickly. For example, the user may complete the back-and-forth gesture or the flicking motion within 0.5 seconds or less according to an exemplary embodiment. Other gestures performed with the scan system 110 may also be used to select an icon, interact with the GUI, or select a point according to other embodiments.

In an embodiment, the probe 120 may be rotated about a longitudinal axis in order to acquire 2D data along a plurality of planes. After placing the probe 120 in the target image area, the user can rotate/move the scan system 110. The motion sensing system 119 detects these movements and communicates the same to the processor 117. The processor 117 (shown in FIG. 1) may use data from the motion sensing system 119 (shown in FIG. 1) to determine how much the probe 120 has to be rotated in order to generate volumetric data. According to an embodiment, it may be necessary to rotate the probe 120 through at least 180 degrees in order to acquire complete volumetric data for a given volume. To achieve this, the user may rotate the scan system 180 degrees. The processor may then use the position and orientation data of each of the planes to generate volumetric data. Similarly, acquiring an image with an extended field of view may be performed by titling the scan system. This amount of tilt of the scan system is mapped to the required amount of tilt by the probe and the motion control system 122 can automatically tilt the probe to the desired amount. The processor 117 may automatically tag each of the 2D frames of data in a buffer or memory as part of a volume in response to detecting each tilt or movement.

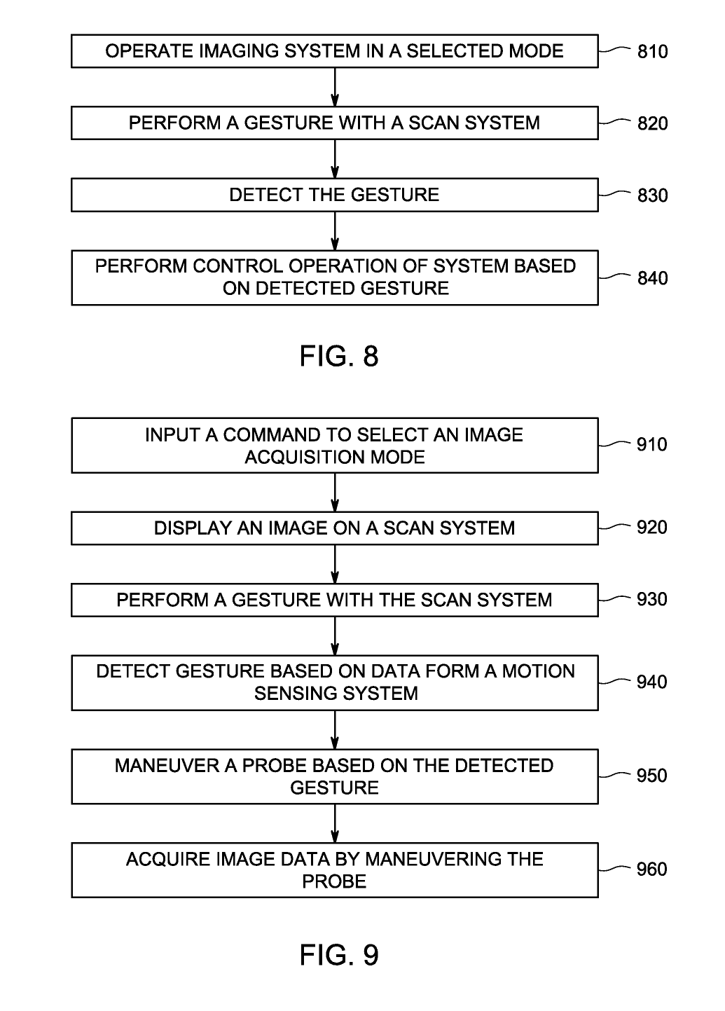

FIG. 8 shows a method of controlling an ultrasound imaging system in accordance with an embodiment. A mode of operation is selected for the imaging system 810. The imaging system could be operated at least in an image acquisition mode or in an image processing mode. The user performs a gesture with the scan system 820. This gesture will help the clinician to maneuver the probe in an image acquisition mode and will help in image processing during the image processing mode. The gestures performed with the scan system are detected by a motion sensing system 830. These gestures are converted into probe movement control instructions in am image acquisition mode and communicated to the probe. In an image processing mode these gestures are translated to user input instructions to process the image data. In the image acquisition mode, gestures of the scan system are mapped to probe movement instructions and in the image processing mode the gestures are converted to user input instructions on image processing. The control operations are performed based on the detected gesture 840. In the image acquisition mode the probe movements are controlled and in the image processing mode, the scan system or the image processing parameters are controlled. For example, based on the gesture, the scan system can control operations like selecting an imaging mode, changing the scan parameters, freeze/unfreeze image, store/print image, etc. The example need not be limited to these. During the image processing mode, either the scan system or various steps in processing of the image data could be controlled by the detected gestures.

FIG. 9 shows a method of controlling an ultrasound imaging system in accordance with an embodiment. A command is given to select a mode of operation of the imaging system 910. In an embodiment, the image acquisition mode is selected. An image is displayed on a display device. In an image acquisition mode, an initial image is displayed on the screen 920. In order to maneuver the probe to get a clearer image, the probe's position and orientation may need to be adjusted slightly. The user performs a gesture with the scan system 930. This gesture will help the clinician to maneuver the probe in an image acquisition mode. The gestures are detected by a motion sensing system associated with the scan system. The gestures are converted to probe control instructions in an image acquisition mode 940. These control instructions are provided to the probe and the probe can be automatically maneuvered by the motion control system associated with the probe 950. Image data is acquired by moving the probe appropriately 960.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of controlling an ultrasound imaging system, the method comprising:
operating the imaging system in a selected mode of operation;
performing a gesture with a scan system;
detecting the gesture based on data from a motion sensing system in the scan system, wherein the motion sensing system includes at least one sensor selected from the group consisting of an accelerometer, a gyro sensor, and a magnetic sensor; and
performing at least one control operation of the imaging system based on the detected gesture in each mode of operation of the imaging system.

2. The method of claim 1, wherein the imaging system is operating in an image acquisition mode and a probe is being positioned on an imaging area.

3. The method of claim 2, wherein detecting the gesture in the image acquisition mode comprises translating gestures of the scan system to control operation instructions for the probe.

4. The method of claim 2, wherein the method further comprises mapping the motion sensing system data to corresponding probe movement to perform probe control operations.

5. The method of claim 2, wherein performing at least one control operation of the probe comprises providing a motion control system adapted to be connected to the probe for implementing the control operation instructions.

6. The method of claim 5, wherein performing at least one control operation of the probe comprises providing a communication channel configured to communicate the control instructions to the probe or to the motion control system.

7. The method of claim 5, wherein performing at least one control operation of the probe comprises adjusting a head of the probe based on the detected gesture.

8. The method of claim 5, wherein performing at least one control operation of the probe comprises adjusting the direction or intensity of a beam from the probe.

9. The method of claim 1, further comprising:
operating the imaging system in an image processing mode;
wherein at least one of functionality of the scan system or the steps in image processing are controlled using gestures detected based on data from the motion sensing system in the scan system.

10. A method of controlling an ultrasound imaging system, the method comprising:
inputting a command to select a mode of operation;
displaying an image on a scan system;
performing a gesture with the scan system;
detecting the gesture based on data from a motion sensing system associated with the scan system, wherein the motion sensing system includes at least one sensor selected from a group consisting of an accelerometer, a gyro sensor, and a magnetic sensor;
maneuvering a probe based on the detected gesture; and
acquiring an image data by maneuvering the probe.

11. The method of claim 10, wherein detecting gestures comprises:
translating gestures of the scan system to control operation instructions for the probe; and
communicating the instructions through a communication channel to the probe.

12. The method of claim 10, further comprises operating the imaging system in an image processing mode.

13. The method of claim 12, further comprising:
detecting gestures of the scan system; and
converting the gestures to predefined user input instructions for processing the image data acquired.

14. An ultrasound imaging system comprising:
a probe, wherein the probe comprises:
a movable head,
at least one transducer element disposed in the head, and
a motion control system configured to control at least the head or the transducer element; and
a scan system in communication with the probe, wherein the scan system comprises:
a housing;
a display;
a motion sensing system attachable to the display or to the housing; and a processor, wherein the processor is configured to receive data from the motion sensing system and to interpret the data as a gesture, and translate the gestures to probe control instructions in a first mode of operation of the imaging system.

15. The ultrasound imaging system of claim 14, wherein the motion sensing system comprises at least one sensor selected from the group consisting of a magnetic sensor, an accelerometer, and a gyro sensor.

16. The ultrasound imaging system of claim 14, further comprising a hand-held ultrasound imaging system.

17. The ultrasound imaging system of claim 14, wherein the processor is configured to perform the control operation of the probe based on the gesture in a first mode of operation and the processor is configured to perform image processing instructions in a second mode of operation based on the gestures of the scan system.

18. The ultrasound imaging system of claim 17, wherein the motion sensing system comprises at least one sensor selected from the group consisting of a magnetic sensor, an accelerometer, a gyro sensor and a camera.

19. The ultrasound imaging system of claim 17, wherein the processor further comprises a memory for storing predefined user input instructions for processing the image data corresponding to the gestures of the scan system.
